(19) United States
(12) Patent Application Publication
(10) Pub. No.: US 2022/0093238 A1
(43) Pub. Date: Mar. 24, 2022

(54) SYSTEMS AND METHODS FOR REMOTE, VIRTUAL, IN-HOME, AND HYBRID VIRTUAL-IN-HOME VETERINARY MEDICINE

(71) Applicant: BETTER VET LLC, Hingham, MA (US)

(72) Inventors: Bruce HERZFELDER, Westwood, MA (US); Zihuai LIU, Revere, MA (US); Carolina GONCEBAT, Boston, MA (US); Michael BORSARI, Fairfield, CT (US); Lucas GREVERS, Westport, CT (US); Federico DORFMAN, Buenos Aires (AR)

(73) Assignee: BETTER VET LLC, Hingham, MA (US)

(21) Appl. No.: 17/478,052

(22) Filed: Sep. 17, 2021

Related U.S. Application Data
(60) Provisional application No. 63/080,203, filed on Sep. 18, 2020.

(51) Int. Cl. G16H 40/20; G16H 80/00; G16H 40/67

(52) U.S. Cl. CPC G16H 40/20; G16H 40/67; G16H 80/00

(57) ABSTRACT

The present disclosure provides systems and methods for remote, virtual, in-home, or hybrid virtual-in-home veterinary medicine.

### CROSS-REFERENCE TO RELATED APPLICATIONS

This application claims priority to U.S. Provisional Patent Application Ser. No. 63/080,203, entitled “SYSTEMS AND METHODS FOR REMOTE, VIRTUAL, IN-HOME, AND HYBRID VIRTUAL-IN-HOME VETERINARY MEDICINE” filed on Sep. 18, 2021. The contents of the aforementioned application are incorporated herein by reference in their entirety.

### BACKGROUND

Modern veterinary practice requires that animals be taken to a clinic or hospital, where the veterinary service is carried out. However, clinic and hospital environments often cause stress and anxiety to the animals. Where the animal is sick or the animals behavior indicates some possible underlying condition, a stressful environment may not provide the best location for performing a checkup or administering veterinary services. For example, performing routine checkups in such a stressful environment may skew indicators used to diagnose illness or track the general health and well being of the animal. Further still, continued monitoring and subsequent checkups for animals may not be carried out due to the inconvenience of repeatedly taking the animal to the clinic and due to the desire to avoid repeated stress of the animal. Where the animal is recovering from an illness or injury, the desire to avoid repeatedly stressing the animal may lead to less than optimal care from a veterinarian.

### BRIEF SUMMARY

As introduced above, in clinic veterinary medicine may provide a less than optimal setting for performing routine checks, treating illness and injury, or performing followup visits due to the fact that many animals exhibit stress or anxiety from the veterinary clinic or hospital environment. Accordingly, there remains a need to provide systems and methods that facilitate remote, virtual, in-home, or hybrid virtual-in-home veterinary medicine.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a pet owner computing device 100 in accordance with embodiment(s) of the disclosure.

FIG. 2 illustrates a vet service provider computing device 200 in accordance with embodiment(s) of the disclosure.

FIG. 3 illustrates a system 300 in accordance with embodiment(s) of the disclosure.

FIG. 4 illustrates a technique 400 in accordance with embodiment(s) of the disclosure.

FIG. 5A illustrates UI elements in accordance with embodiment(s) of the disclosure.

FIG. 5B illustrates UI elements in accordance with embodiment(s) of the disclosure.

FIG. 6 illustrates a technique 600 in accordance with embodiment(s) of the disclosure.

FIG. 7A illustrates UI elements in accordance with embodiment(s) of the disclosure.

FIG. 7B illustrates UI elements in accordance with embodiment(s) of the disclosure.

FIG. 8 illustrates a technique 800 in accordance with embodiment(s) of the disclosure.

FIG. 9A illustrates UI elements in accordance with embodiment(s) of the disclosure.

FIG. 9B illustrates UI elements in accordance with embodiment(s) of the disclosure.

FIG. 9C illustrates UI elements in accordance with embodiment(s) of the disclosure.

FIG. 9D illustrates UI elements in accordance with embodiment(s) of the disclosure.

FIG. 9E illustrates UI elements in accordance with embodiment(s) of the disclosure.

FIG. 9F illustrates UI elements in accordance with embodiment(s) of the disclosure.

FIG. 9G illustrates UI elements in accordance with embodiment(s) of the disclosure.

FIG. 9H illustrates UI elements in accordance with embodiment(s) of the disclosure.

FIG. 9I illustrates UI elements in accordance with embodiment(s) of the disclosure.

FIG. 9J illustrates UI elements in accordance with embodiment(s) of the disclosure.

FIG. 10 illustrates a system 1000 in accordance with embodiment(s) of the disclosure.

FIG. 11 illustrates a computer-readable storage medium 1100 in accordance with one embodiment.

FIG. 12 illustrates a diagrammatic representation of a machine 1200 in the form of a computer system within which a set of instructions may be executed for causing the machine to perform any one or more of the methodologies discussed herein, according to an example embodiment.

### DETAILED DESCRIPTION

As noted, the present disclosure is directed to systems and methods for providing veterinary services remotely, virtually, in-home, and hybrid virtual-in-home.

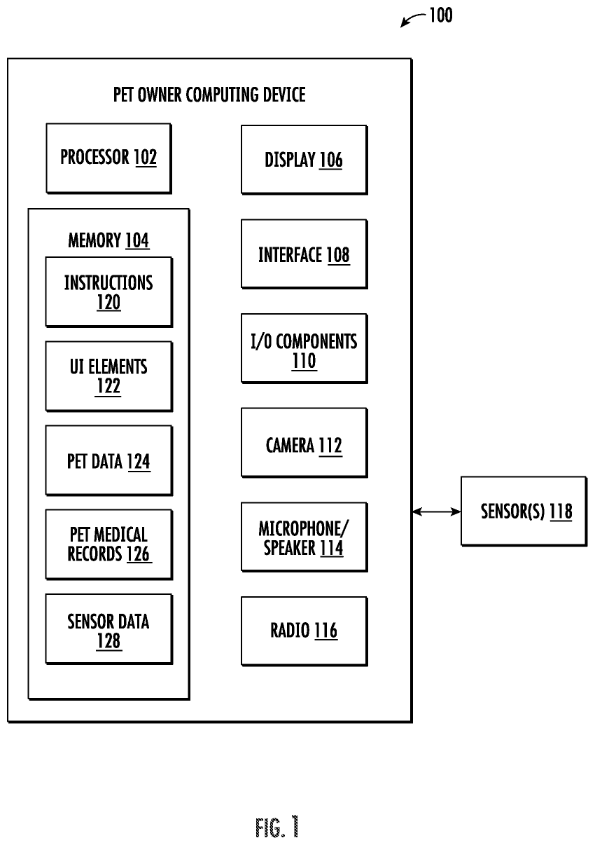

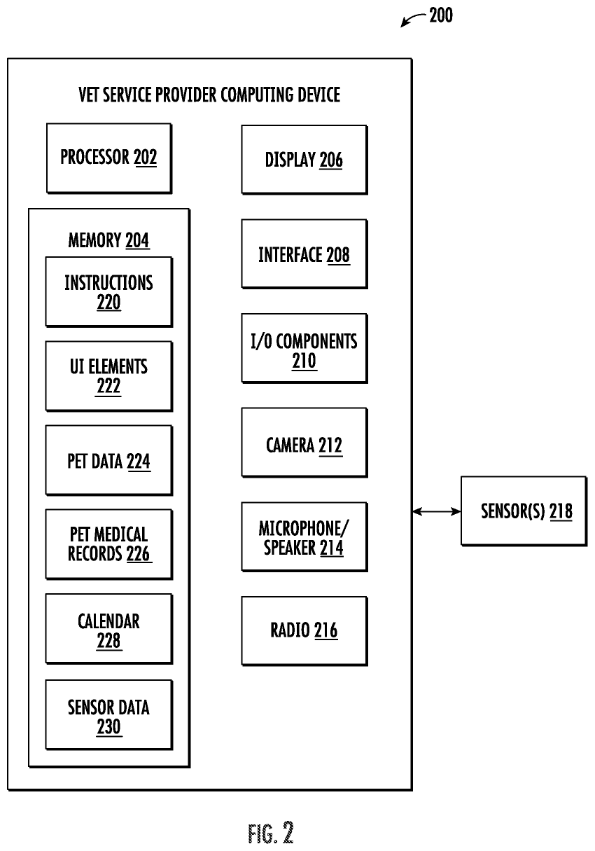

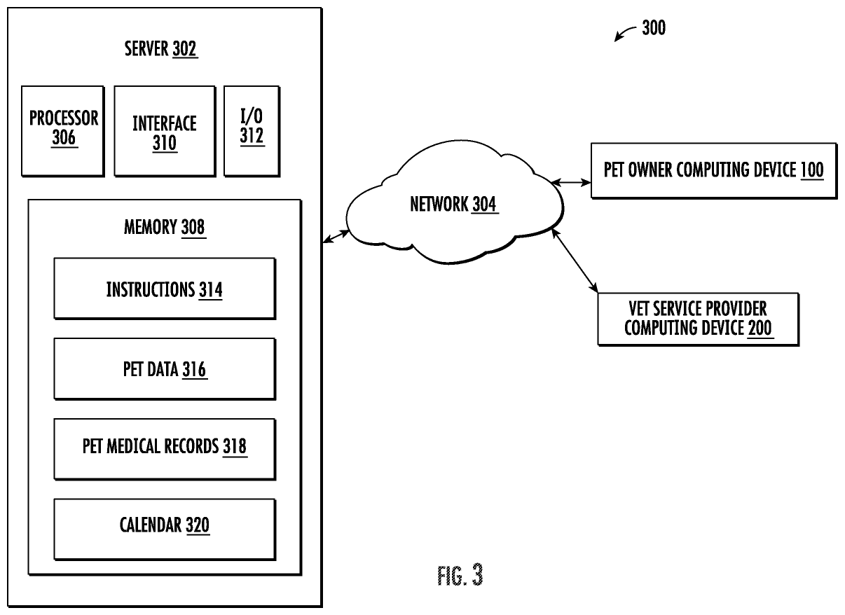

FIG. 1, FIG. 2, and FIG. 3 illustrate devices and a system arranged to cooperate to facilitate remote, virtual, in-home, or hybrid virtual-in-home veterinary medicine. Turning more particularly to FIG. 1, which illustrates an example pet owner computing device 100 (often simply device 100), which can be a computing device owned and/or operated by an owner of a pet that is to receive veterinary medicine as detailed herein. In general, device 100 can be any of a variety of computing devices, such as, a smart phone, a tablet computer, a laptop computer, a desktop computer, a smart watch, a smart pet collar, or the like. Device 100 includes a processor 102, a memory 104, a display 106, an interface 108, input and output (I/O) components 110, camera 112, microphone and speaker 114, and radio 116. Device 100 may optionally also include, or be operably coupled to, one or more sensors 118.

The memory 104 may store instructions 120 executable by processor 102, which when executed cause processor 102 and device 100 to implement or carry out features and functions detailed herein. Memory 104 may further store UI elements 122, pet data 124, pet medical records 126, and sensor data 128. With some examples, processor 102 can execute instructions 120 to generate user interface (UI) elements 122 and pet data 124. Additionally, processor 102 can execute instructions 120 to retrieve pet medical records 126. Furthermore, processor 102 can execute instructions 120 to receive sensor data 128 from sensor 118.

Device 100 can be used to facilitate other embodiments of the present disclosure, such as, for example, a remote, virtual, in-home, or hybrid virtual-in-home veterinary appointment, including selecting the appointment, scheduling the appointment, conducting the appointment, following up on the appointment (e.g., ordering prescriptions, or the like). These, and other embodiments, are described in greater detail below.

With some examples, the processor 102 may include circuity or processor logic, such as, for example, any of a variety of commercial processors. In some examples, the processor 102 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 102 may include graphics processing portions and may include dedicated memory, multiple-threaded processing and/or some other parallel processing capability.

The memory 104 may include logic, a portion of which includes arrays of integrated circuits, forming non-volatile memory to persistently store data or a combination of non-volatile memory and volatile memory. It is to be appreciated, that the memory 104 may be based on any of a variety of technologies. In particular, the arrays of integrated circuits included in memory 104 may be arranged to form one or more types of memory, such as, for example, dynamic random access memory (DRAM), NAND memory, NOR memory, or the like.

Display 106 can be based on any of a variety of display technologies, such as, for example, a cathode ray tube (CRT), a liquid crystal display (LCD), plasma display, light emitting diode (LED) display, or an organic light emitting diode (OLED) display. With some examples, display 106 can be a touch sensitive display. It is noted, display 106 may be external to device 100, such as, for example, embodied as a computer monitor or television and coupled to device 100 via any of a variety of display data interfaces.

Interface 108 may include logic and/or features to support a communication interface. For example, the interface 108 may include one or more interfaces that operate according to various communication protocols or standards to communicate over direct or network communication links. Direct communications may occur via use of communication protocols or standards described in one or more industry standards (including progenies and variants). For example, the interface 108 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like.

The I/O components 110 may include one or more components to provide input to or to provide output from the device 100. For example, the I/O components 110 may be a keyboard (hardware, virtual, etc.), mouse, joystick, track pad, button, touch layers of a display, haptic feedback device, or the like.

Camera 112 as well as microphone and speaker 114 may be any of a variety of cameras, microphone, and speakers used for teleconference and video conference applications. Camera 112 and microphone and speaker 114 may be integrated into device 100 or may be external to device 100.

The radio 116 may include circuitry arranged to communicate data with one or more other devices (see FIG. 3) via any of a variety of communication protocols. Such communication may involve communication across one or more networks, such a wireless local area networks (WLAN) and/or cellular network. In some examples, radio 116 can be arranged to communicate via Wi-Fi, Bluetooth, Zigbee, LTE, 5G, RFID, or the like.

The sensor 118 (or sensors 118) may include a number of any of a variety of sensors arranged to detect information, such, as, physical surrounding information, geo-information, biometric information, or the like. For example, sensor 118 can include a thermometer, a heart rate sensor, a digital stethoscope, a digital otoscope, a digital ophthalmoscope, a blood pressure monitor, a tonopen, an ultrasound, a centrifuge, a cold laser, a multiscope (e.g., touch screen multiscope with telemedicine capabilities, or the like), an x-ray, a portable lab device, a refractometer, or the like. Signals from sensor 118 can be used to capture indications of lab results, biometric indications, or the like of a pet. It is noted, that sensor 118 could be located externally to device 100. For example, sensor 118 could be embodied in a smart pet collar, a microchip implanted in a pet, or the like. Such a sensor 118 could be wirelessly coupled to device 100.

FIG. 2 illustrates vet service provider computing device 200 (often simply device 200), which can be a computing device owned and/or operated by a veterinary medicine service provider. During use, device 200 may be operated by an administrator of a veterinary service provider, a veterinary technician, a nurse, or a veterinarian. In general, device 200 can be any of a variety of computing devices, such as, a smart phone, a tablet computer, a laptop computer, a desktop computer, or the like. Device 200 includes, at least, a processor 202, a memory 204, a display 206, an interface 208, I/O components 210, camera 212, microphone and speaker 214, and radio 116. Device 200 may optionally also include, or be operably coupled to, one or more sensors 218.

The memory 204 may store instructions 220 executable by processor 202, which when executed cause processor 202 and device 200 to implement or carry out features and functions detailed herein. Memory 204 may further store UI elements 222, pet data 224, pet medical records 226, calendar 228, and sensor data 230. With some examples, processor 202 can execute instructions 220 to generate user interface (UI) elements 222 and pet medical records 226. Additionally, processor 202 can execute instructions 220 to retrieve pet data 224 and calendar 228. Furthermore, processor 202 can execute instructions 220 to receive sensor data 230 from sensor 218. In some examples, sensor data 230 can be received from device 100 (e.g., via server 302, or the like).

Device 200 can be used to facilitate other embodiments of the present disclosure, such as, for example, a remote, virtual, in-home, and/or hybrid virtual-in-home veterinary appointment, including identifying the appointment scheduled via device 100, conducting the appointment, documenting the appointment, etc. These, and other embodiments, are described in greater detail below.

With some examples, the processor 202 may include circuity or processor logic, such as, for example, any of a variety of commercial processors. In some examples, the processor 202 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 202 may include graphics processing portions and may include dedicated memory, multiple-threaded processing and/or some other parallel processing capability.

The memory 204 may include logic, a portion of which includes arrays of integrated circuits, forming non-volatile memory to persistently store data or a combination of non-volatile memory and volatile memory. It is to be appreciated, that the memory 204 may be based on any of a variety of technologies. In particular, the arrays of integrated circuits included in memory 204 may be arranged to form one or more types of memory, such as, for example, dynamic random access memory (DRAM), NAND memory, NOR memory, or the like.

Display 206 can be based on any of a variety of display technologies, such as, for example, a cathode ray tube (CRT), a liquid crystal display (LCD), plasma display, light emitting diode (LED) display, or an organic light emitting diode (OLED) display. With some examples, display 206 can be a touch sensitive display. It is noted, display 206 may be external to device 200, such as, for example, embodied as a computer monitor or television and coupled to device 200 via any of a variety of display data interfaces.

Interface 208 may include logic and/or features to support a communication interface. For example, the interface 208 may include one or more interfaces that operate according to various communication protocols or standards to communicate over direct or network communication links. Direct communications may occur via use of communication protocols or standards described in one or more industry standards (including progenies and variants). For example, the interface 208 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like.

The I/O components 210 may include one or more components to provide input to or to provide output from the device 100. For example, the I/O components 210 may be a keyboard (hardware, virtual, etc.), mouse, joystick, track pad, button, touch layers of a display, haptic feedback device, or the like.

Camera 212 as well as microphone and speaker 214 may be any of a variety of cameras, microphone, and speakers used for teleconference and video conference applications. Camera 212 and microphone and speaker 214 may be integrated into device 200 or may be external to device 200.

The radio 216 may include circuitry arranged to communicate data with one or more other devices (see FIG. 3) via any of a variety of communication protocols. Such communication may involve communication across one or more networks, such a wireless local area networks (WLAN) and/or cellular network. In some examples, radio 216 can be arranged to communicate via Wi-Fi, Bluetooth, Zigbee, LTE, 5G, or the like.

The sensor 218 (or sensors 218) may include a number of any of a variety of sensors arranged to detect information, such, as, physical surrounding information, geo-information, biometric information, or the like. For example, sensor 218 can include a radar sensor, infrared sensors, light sensors, RFID sensors, gyroscopes, a global positioning sensors (GPS), a heart rate sensor, a temperature sensor, or the like. Signals from sensor 218 can be used to. It is noted, that sensor 218 could be located externally to device 200.

The sensor 218 (or sensors 218) may include a number of any of a variety of sensors arranged to detect information, such, as, physical surrounding information, geo-information, biometric information, or the like. For example, sensor 218 can include a thermometer, a heart rate sensor, a digital stethoscope, a digital otoscope, a digital ophthalmoscope, a blood pressure monitor, a tonopen, an ultrasound, a centrifuge, a cold laser, a multiscope (e.g., touch screen multiscope with telemedicine capabilities, or the like), an x-ray, a portable lab device, a refractometer, or the like. Signals from sensor 218 can be used to capture indications of lab results, biometric indications, or the like of a pet. It is noted, that sensor 218 could be located externally to device 200. In other examples, device 200 could be communicatively connected to sensor 118 of device 100 (e.g., via server 302 and network 304, or the like).

FIG. 3 illustrates an example system 300 including device 100, device 200, and server 302. In system 300, device 100, device 200, and server 302 are communicatively coupled via network 304. Network 304 could be, for example, a local area network (LAN), a wide area network (WAN), or a cellular network (e.g., LTE, 3GPP, or the like). In some embodiments, network 304 could include the Internet.

The server 302 can include a processor 306, memory 308, interface 310, and I/O components 312. The memory 308 may store instructions 314, pet data 316, UI elements 318, and calendar 320. With some examples, processor 306 can execute instructions 314 to generate receive and/or provide pet data 316 and pet medical records 318. Additionally, processor 306 can execute instructions 314 to coordinate scheduling of a remote, virtual, in-home, and/or hybrid virtual-in-home veterinary service via interaction with device 100. Likewise, processor 306 can execute instructions 314 to provide details (e.g., via calendar 320) of a scheduled remote, virtual, in-home, and/or hybrid virtual-in-home veterinary service to device 200. Server 302 within system 300 can be used to facilitate other embodiments of the present disclosure, such as, for example, a remote, virtual in-home, and/or hybrid virtual-in-home veterinary appointments. These, and other embodiments, are described in greater detail below.

With some examples, the processor 306 may include circuity or processor logic, such as, for example, any of a variety of commercial processors. In some examples, the processor 306 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 306 may include graphics processing portions and may include dedicated memory, multiple-threaded processing and/or some other parallel processing capability.

Memory 308 may include logic, a portion of which includes arrays of integrated circuits, forming non-volatile memory to persistently store data or a combination of non-volatile memory and volatile memory. It is to be appreciated, that the memory 308 may be based on any of a variety of technologies. In particular, the arrays of integrated circuits included in memory 308 may be arranged to form one or more types of memory, such as, for example, dynamic random access memory (DRAM), NAND memory, NOR memory, or the like.

Interface 310 may include logic and/or features to support a communication interface. For example, the interface 310 may include one or more interfaces that operate according to various communication protocols or standards to communicate over direct or network communication links. Direct communications may occur via use of communication protocols or standards described in one or more industry standards (including progenies and variants). For example, the interface 310 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, interface 310 can be a network interface arranged to couple server 302 to a network (e.g., network 304).

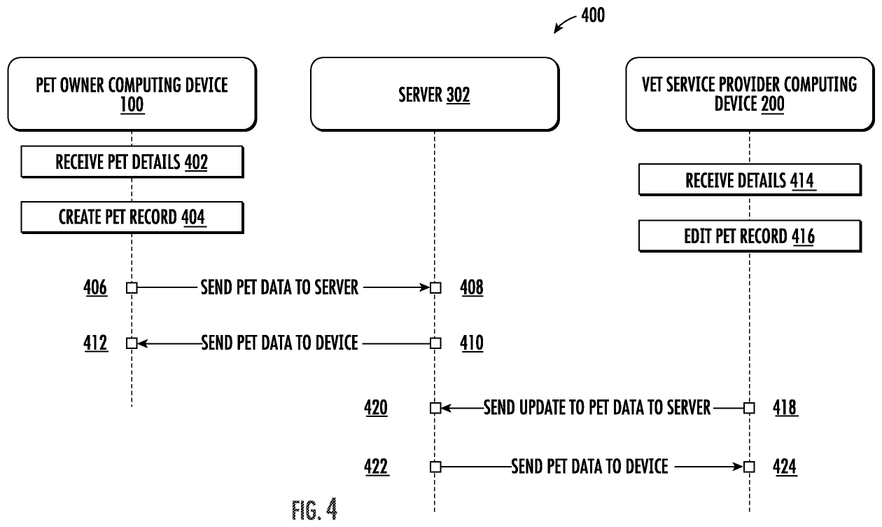

As noted, system 300 can facilitate numerous embodiments to provide remote, virtual, in-home, and/or hybrid virtual-in-home veterinary medicine. Operation and features of system 300 are described with reference other figures depicted and described herein, such as, for example, FIG. 4.

In general, FIG. 4 depicts a technique 400 to initiate or prepare a system for facilitation of remote, virtual, in-home, or hybrid virtual-in-home veterinary services. It is noted, technique 400 is described with reference to the system 300 of FIG. 3 including server 302, vet service provider computing device 200, and pet owner computing device 100. This is done for purposes of convenience and clarity, as opposed to limitation. For example, technique 400 could be implemented by a system having a different arrangement or entities from that of the system 300 of FIG. 3.

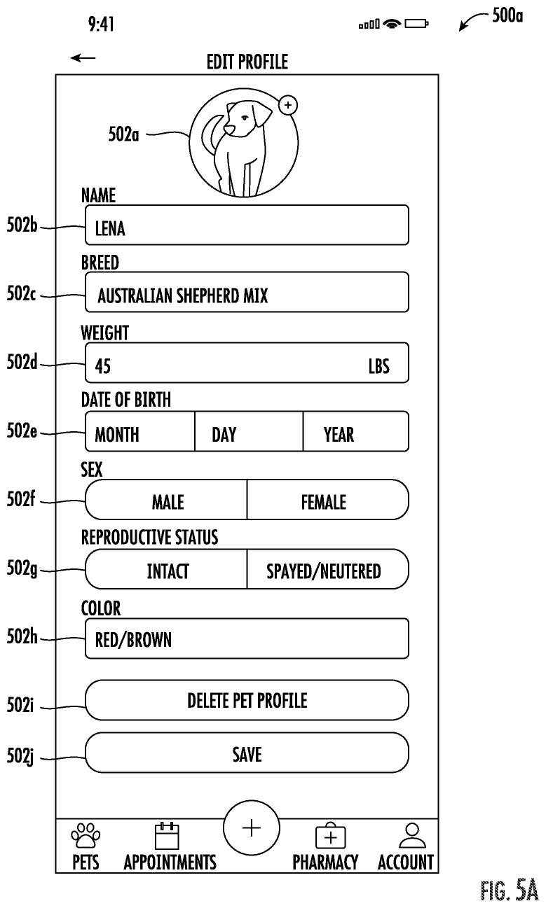

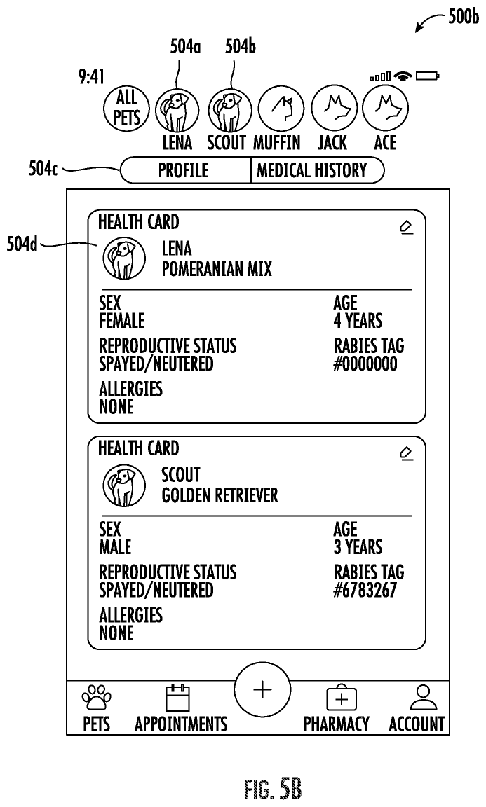

Technique 400 can begin at block 402. At block 402, device 100 can receive information about a pet for which veterinary services are desired. For example, processor 102 can execute instructions 120 to receive indications of details (e.g., demographic data, or the like) of a pet for which veterinary services are desired. As a specific example, processor 102 can execute instructions 120 to generate UI elements 122 (e.g., including text input boxes, check boxes, selection bubbles, drop down boxes, or the like) for display on display 106. Responsive to displaying UI elements 122, processor 102 can execute instructions 120 to receive input via UI elements 122. Example UI elements, which can be UI elements 122, for receiving pet data at block 402 is depicted in FIG. 5A.

Continuing to block 404, device 100 can generate pet data 124. For example, processor 102 can execute instructions 120 to generate pet data 124 responsive to receiving pet information at block 402. Pet data 124 can be any of a variety of information, such as, for example, information depicted in example UI elements, which can be UI elements 122, for displaying indications of pet data 124 is depicted in FIG. 5B.

Technique 400 can further include operations for sending an information element including indications of pet data 124 to server 302. For example, technique 400 can include block 406 and block 408 where device 100 sends an information element including indications of pet data 124 to server 302 and server 302 receives the information element. Likewise, technique 400 can include block 410 and block 412 where server 302 sends an information element including indications of pet data 124 to device 100 and device 100 receives the information element. In some examples, block 410 can be executed by server 302 (e.g., processor 306 in executing instructions 314, or the like) responsive to receiving a request for pet data 124 from device 100.

Technique 400 can also include block 414. At block 414, device 200 can receive information about a pet for which veterinary services are to be provided or have been provided. Said differently, at block 414, device 200 can receive information about a pet patient, for example, by a nurse, veterinarian, or the like. For example, processor 202 can execute instructions 220 to receive indications of details (e.g., pet behavior, owner details, or the like) of a pet for which veterinary services are provided or to be provided. As a specific example, processor 202 can execute instructions 220 to generate UI elements 222 (e.g., including text input boxes, check boxes, selection bubbles, drop down boxes, or the like) for display on display 206. Responsive to displaying UI elements 222, processor 202 can execute instructions 220 to receive input via UI elements 222.

Continuing to block 416, device 200 can generate pet data 224. For example, processor 202 can execute instructions 220 to generate pet data 224 responsive to receiving pet information at block 414. Pet data 224 can be any of a variety of information, such as, for example, information depicted in example UI elements, which can be UI elements 122, for displaying indications of pet data 124 is depicted in FIG. 5B. It is noted, pet data 224 can include information private or not available to the owner of the pet. Said differently, pet data 224 can include pet data 124 that is visible to pet owner computing device 100 as well as other pet information (e.g., pet personality, owner personality, interaction details, or the like) that is not visible to pet owner computing device 100. In other examples, pet data 124 and pet data 224 can be the same.

Technique 400 can further include operations for sending an information element including indications of pet data 224 to server 302. For example, technique 400 can include block 418 and block 420 where device 200 sends an information element including indications of pet data 224 to server 302 and server 302 receives the information element. Likewise, technique 400 can include block 422 and block 424 where server 302 sends an information element including indications of pet data 224 to device 200 and device 200 receives the information element. In some examples, block 422 can be executed by server 302 (e.g., processor 306 in executing instructions 314, or the like) responsive to receiving a request for pet data 224 from device 200.

FIG. 5A depicts UI elements 500a which can be generated by a computing device to receive information of a pet for which remote, virtual, in-home, or hybrid virtual-in-home veterinary services are desired. For example, UI elements 500a includes a number of items 502, such as, item 502a, item 502b, item 502c, item 502d, item 502e, item 502f, item 502g, item 502h, item 502i, and item 502j. As depicted, items 502 can be various input options (e.g., text boxes, drop down boxes, text bubbles, image input options, etc.) for receiving information about a pet. Further, ones of items 502 can be used to savings and/or deleting a pet (or a record of a pet). Information indicated in items 502 can be saved as pet data 124. For example, processor 102 can execute instructions 120 to store indications of information of indicated in items 502 as pet data 124.

FIG. 5B depicts UI elements 500b which can be generated by a computing device to display information of a pet for which remote, virtual, in-home, or hybrid virtual-in-home veterinary services are desired and/or have been provided. For example, UI elements 500b includes a number of items 504, such as, item 504a, item 504b, item 504c, and item 504d. As depicted, items 504 can be various display or selection options (e.g., selection boxes, text display elements, image display elements, etc.) for providing information about a pet. In some examples, ones of items 502 can be used to savings and/or deleting a pet (or a record of a pet). Information indicated in items 502 can be saved as pet data 124. For example, processor 102 can execute instructions 120 to store indications of information of indicated in items 502 as pet data 124.

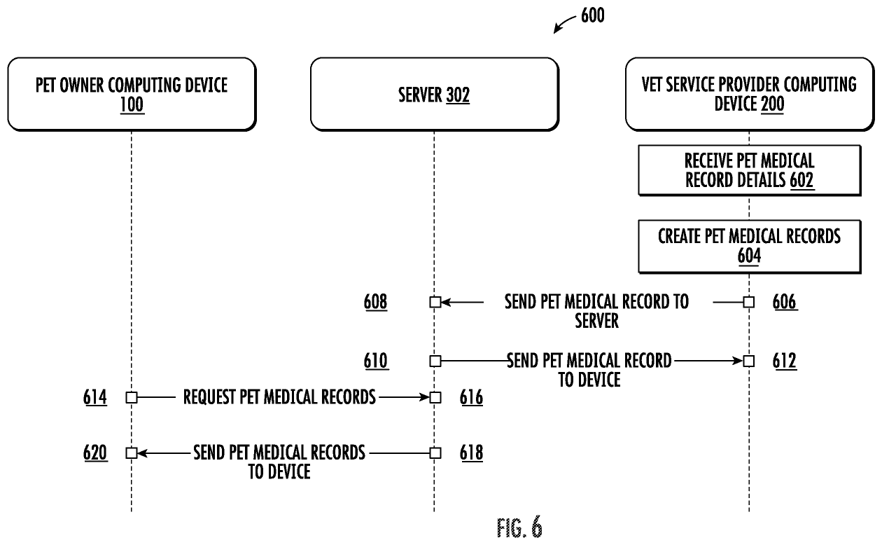

In general, FIG. 6 depicts a technique 600 to create and view pet medical records for remote, virtual, in-home, or hybrid virtual-in-home veterinary services. It is noted, technique 600 is described with reference to the system 300 of FIG. 3 including server 302, vet service provider computing device 200, and pet owner computing device 100. This is done for purposes of convenience and clarity, as opposed to limitation. For example, technique 600 could be implemented by a system having a different arrangement or entities from that of the system 300 of FIG. 3.

Technique 600 can begin at block 602. At block 602, device 200 can receive information about a remote, virtual, in-home, or hybrid virtual-in-home veterinary service (e.g., visit summary, procedures performed, medications administered, location of procedure, type of interaction such as, virtual, in-home, hybrid, etc.). For example, processor 202 can execute instructions 220 to receive indications of details about such a veterinary service.

Continuing to block 604, device 200 can generate pet medical records 226. For example, processor 202 can execute instructions 220 to generate pet medical records 226 responsive to receiving the information at block 602. In general, pet medical records 226 can include any information pertaining to the health or medical history of the pet patient. For example, pet medical records 226 can include information regarding subjective (e.g., lethargic, agitated, etc.) and objective (test results, sensor data 128, sensor data 230, or the like) observations of the pet patient. Furthermore, pet medical records 226 can include other related information (e.g., differential diagnosis, pet demographics, vital signs, diagnoses, medications, treatment plans, progress notes, problems, immunization dates, allergies, radiology images, laboratory and other test results, etc.)

Technique 600 can further include operations for sending an information element including indications of pet medical records 226 to server 302. For example, technique 600 can include block 606 and block 608 where device 200 sends an information element including indications of pet medical records 226 to server 302 and server 302 receives the information element. Likewise, technique 600 can include block 610 and block 612 where server 302 sends an information element including indications of pet medical records 226 to device 200 and device 200 receives the information element. In some examples, block 610 can be executed by server 302 (e.g., processor 306 in executing instructions 314, or the like) responsive to receiving a request for pet medical records 226 from device 200.

Technique 600 can also include operations for sending an information element including indications of pet medical records 126 to device 100. For example, technique 600 can include block 614 and block 616 where device 100 sends a request for pet medical records 126 to server 302 and server 302 receives the request. Likewise, technique 600 can include block 618 and block 620 where server 302 sends an information element including indications of pet medical records 126 to device 100 and device 100 receives the information element. It is noted, that pet medical records 126 can be a redacted version of pet medical records 226. Said differently, pet medical records 226 can include pet medical records 126 that is visible to pet owner computing device 100 as well as other pet medical record information (e.g., pet personality, interaction details, or the like) that is not visible to pet owner computing device 100.

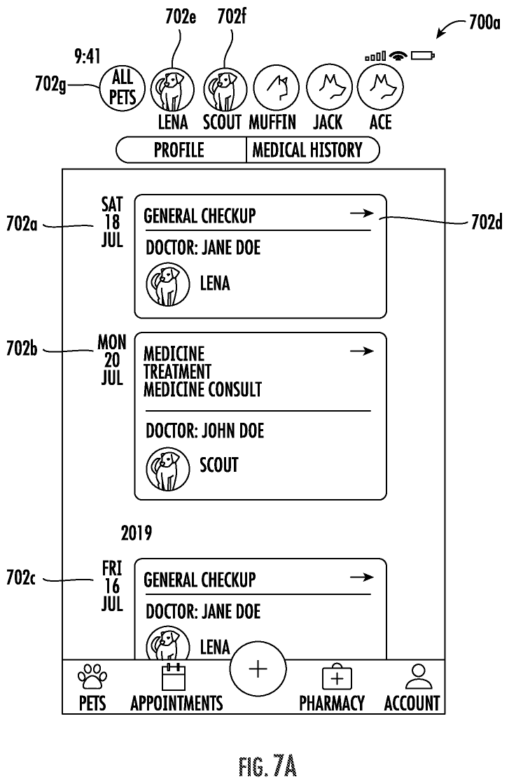

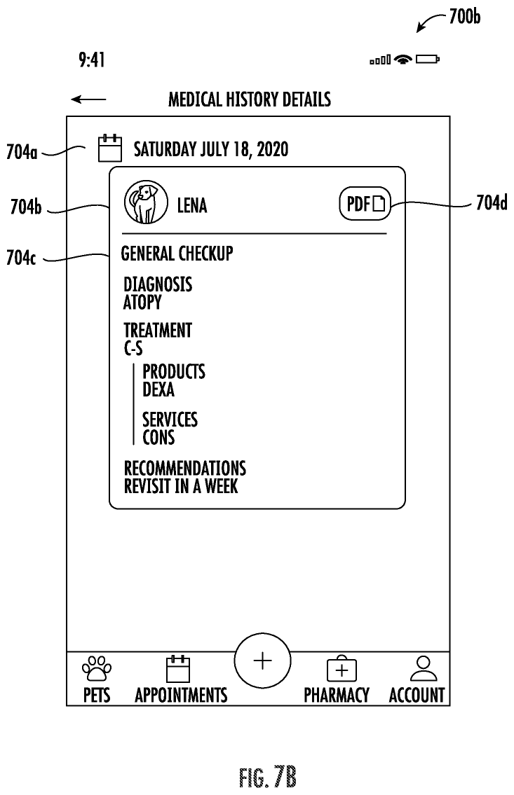

FIG. 7A depicts UI elements 700a which can be generated by a computing device to display pet medical record information for which remote, virtual, in-home, or hybrid virtual-in-home veterinary services have been provided. For example, UI elements 700a includes a number of items 702, such as, item 702a, item 702b, item 702c, item 702d, item 702e, item 702f, and item 702g. As depicted, items 702 can be various display or export items (e.g., text display elements, image display elements, buttons, etc.) for providing information about pet medical records. For example, items 702 include information about a listing of remote, virtual, in-home, or hybrid virtual-in-home veterinary services provided for a particular pet. Processor 102 of device 100, in executing instructions 120, can generate UI elements 122 to include UI elements 700a and cause UI elements 700a to be displayed on display 106. As another example, processor 202 of device 200, in executing instructions 220, can generate UI elements 222 to include UI elements 700a and cause UI elements 700a to be displayed on display 206.

FIG. 7B depicts UI elements 700b which can be generated by a computing device to display pet medical record information for which remote, virtual, in-home, or hybrid virtual-in-home veterinary services have been provided. For example, UI elements 700b includes a number of items 704, such as, item 704a, item 704b, item 704c, and item 704d. As depicted, items 704 can be various display or export items (e.g., text display elements, image display elements, buttons, etc.) for providing information about pet medical records. For example, items 704 include information about a specific remote, virtual, in-home, or hybrid virtual-in-home veterinary service provided on a particular data (e.g., Jul. 18, 2020, or the like). Processor 102 of device 100, in executing instructions 120, can generate UI elements 122 to include UI elements 700a and cause UI elements 700a to be displayed on display 106. As another example, processor 202 of device 200, in executing instructions 220, can generate UI elements 222 to include UI elements 700a and cause UI elements 700a to be displayed on display 206.

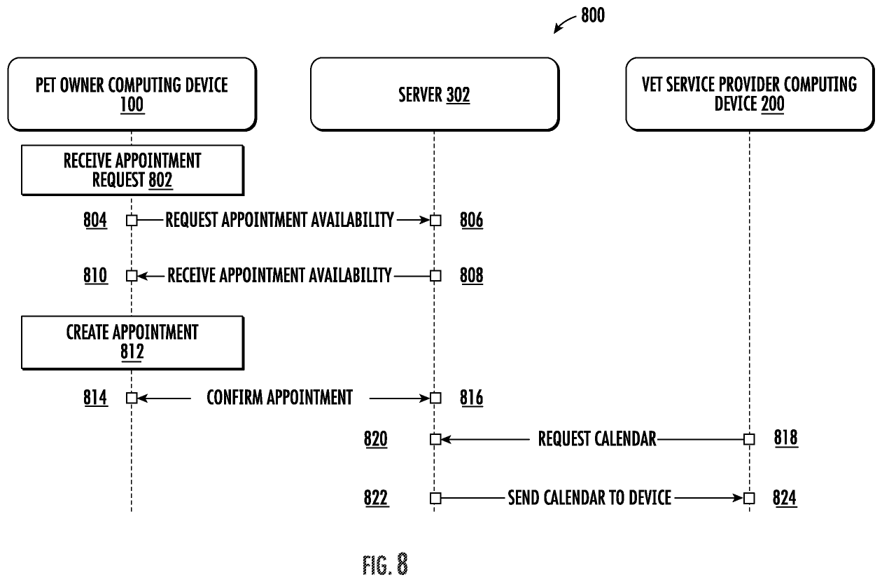

In general, FIG. 8 depicts a technique 800 to schedule (or cancel) remote, virtual, in-home, or hybrid virtual-in-home veterinary services and to provide a calendar of such scheduled services to a pet owner device and service provider device. It is noted, technique 800 is described with reference to the system 300 of FIG. 3 including server 302, vet service provider computing device 200, and pet owner computing device 100. This is done for purposes of convenience and clarity, as opposed to limitation. For example, technique 800 could be implemented by a system having a different arrangement or entities from that of the system 300 of FIG. 3.

Technique 800 can begin at block 802. At block 802, pet owner computing device 100 can receive a request to schedule a remote, virtual, in-home, or hybrid virtual-in-home veterinary service. For example, processor 102 can execute instructions 120 to receive a request to schedule such a service. As a specific example, processor 102 can execute instructions 120 to generate UI elements 122 (e.g., including text input boxes, check boxes, selection bubbles, drop down boxes, or the like) for display on display 106. Responsive to displaying UI elements 122, processor 102 can execute instructions 120 to receive a request for a veterinary service. As a specific example, processor 102 can execute instructions 120 to receive a request to schedule a virtual veterinary service (e.g., video consultation, or the like). As another example, processor 102 can execute instructions 120 to receive a request to schedule an in-home veterinary service (e.g., at the pet owner's residence, at the pet owner's place of work, or the like). In another specific example, processor 102 can execute instructions 120 to receive a request to schedule a hybrid virtual-in-home veterinary service.

As used herein, a hybrid virtual-in-home visit is one where a nurse (or veterinary technician, or the like)) conducts an in-home visit with the pet patient while a veterinarian attends the in-home visit virtually. For example, a hybrid virtual-in-home veterinary visit could be requested for any “wellness” type visits. In a few specific examples, a hybrid virtual-in-home visit could be requested for annual vaccinations (e.g., where a nurse is allowed to administer the vaccination). For example, the nurse can do full evaluation of vital signs (e.g., ears, nose, throat, gums, anal glands, reproductive condition, skin conditions, etc.) and they can visit a pet to administer medicine under doctor's orders.

With some examples, an in-home visit could be dynamically modified to a hybrid virtual-in-home visit where, for example, when nurse conducting an in-home visit finds for any reason, something amiss, the in-home visit could be dynamically modified to a hybrid visit where the veterinary nurse is in-person and a veterinary doctor is virtual to formally diagnose a condition and/or prescribe medicine and treatment protocol.

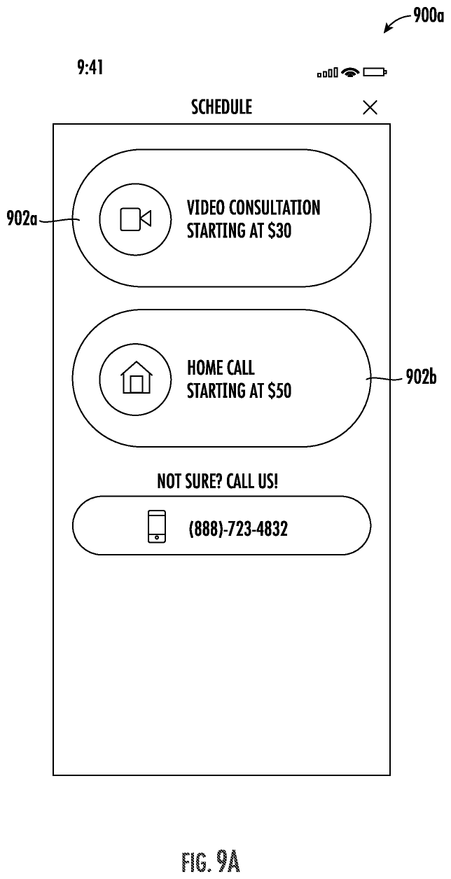

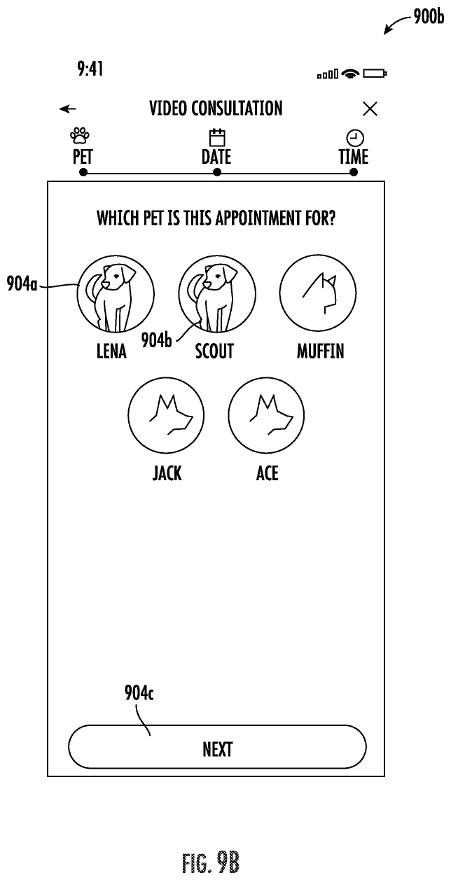

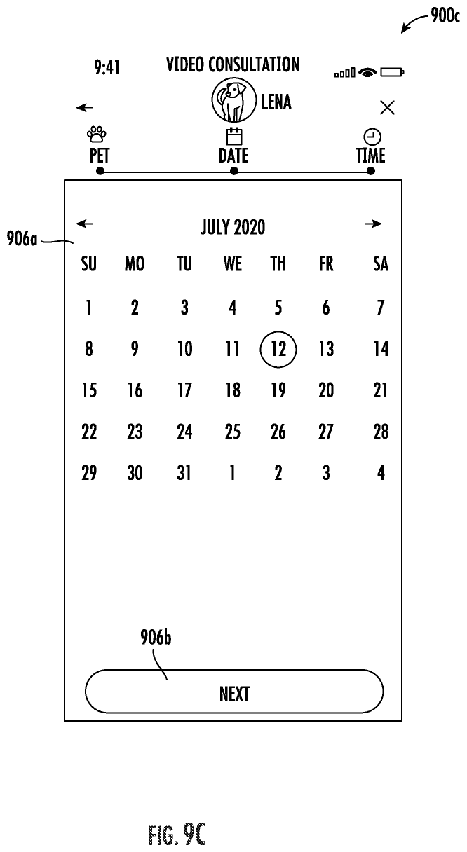

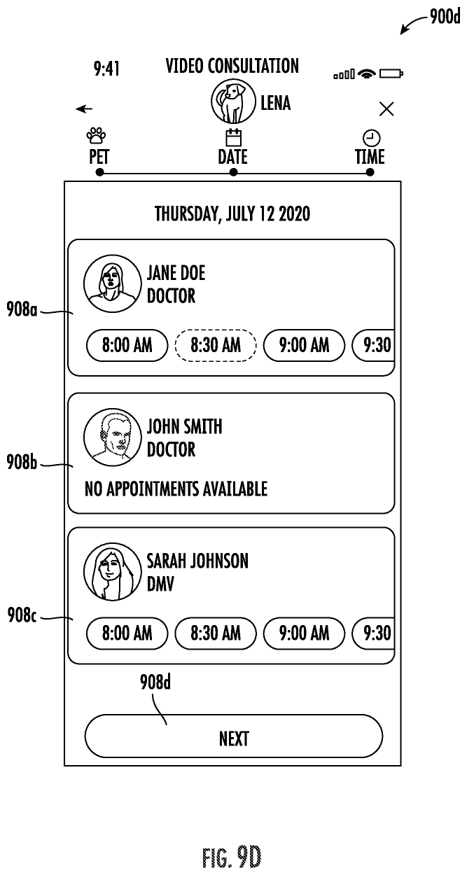

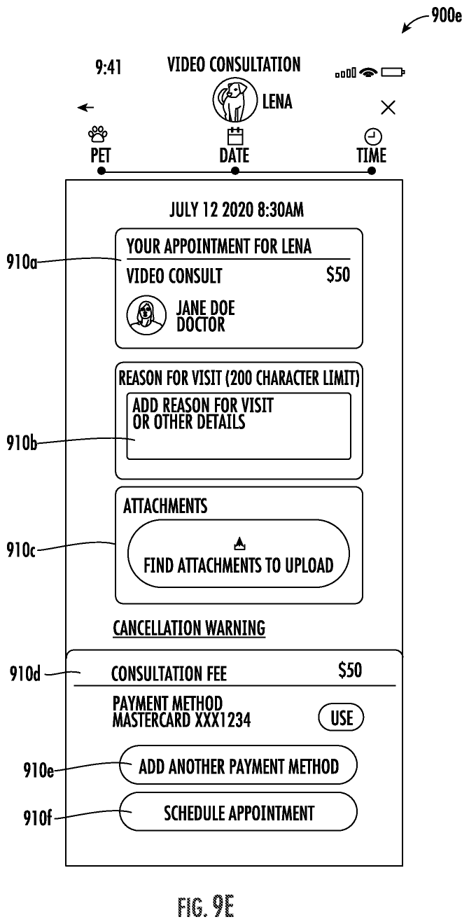

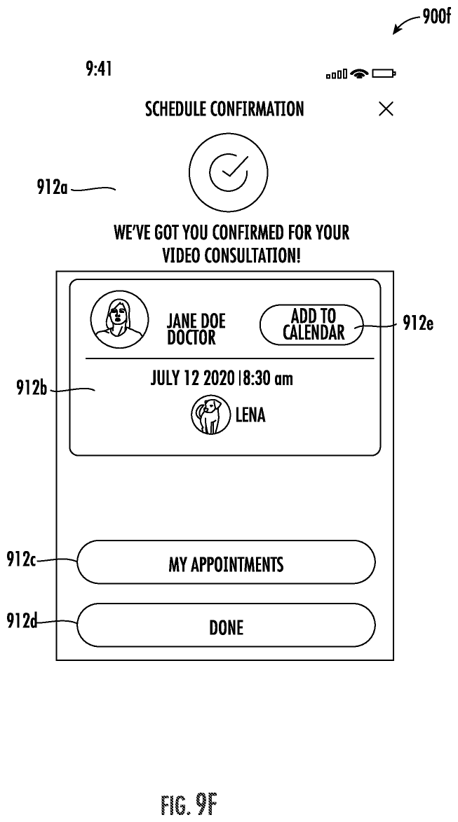

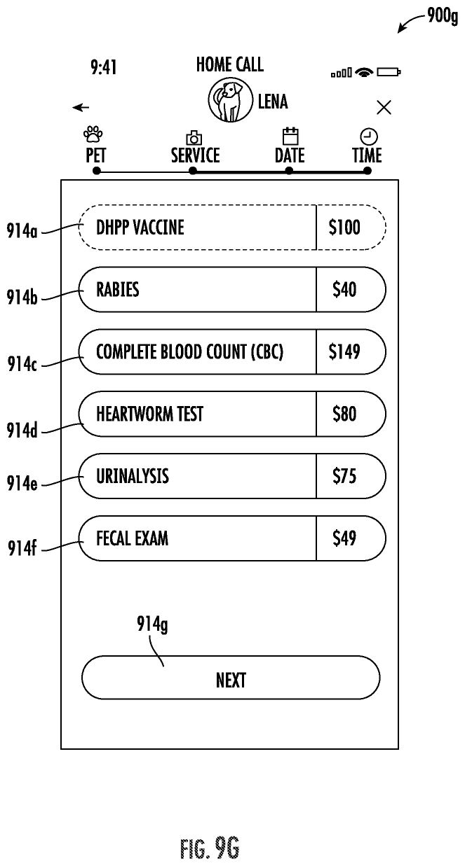

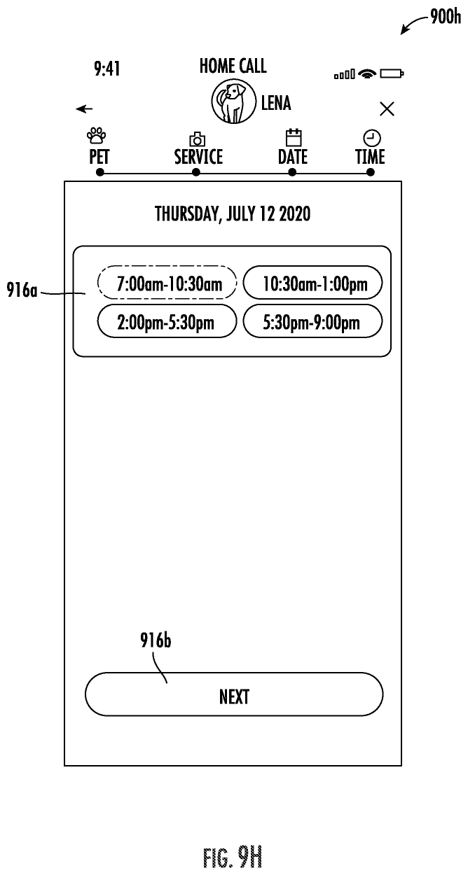

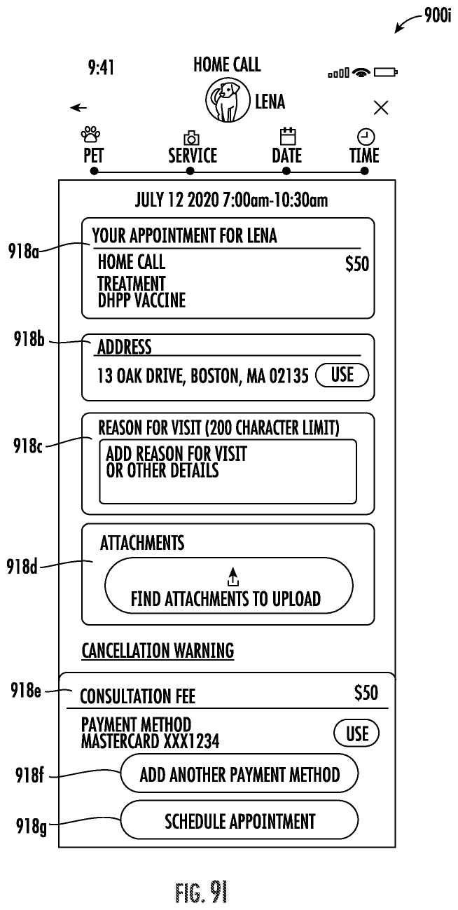

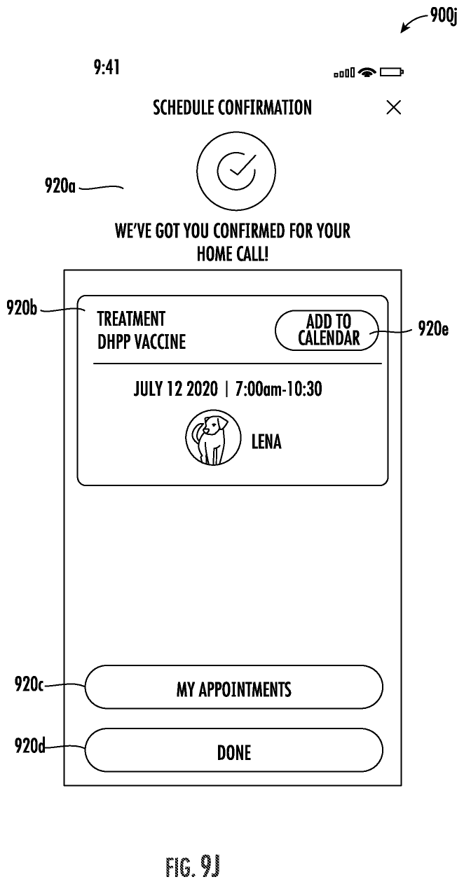

Example UI elements, which can be UI elements 122, for receiving a request to schedule such a service at block 802 is depicted in FIG. 9A. As a further example, UI elements (e.g., UI elements 122) for receiving a request to schedule such a service at block 802 is depicted in FIG. 9B. In a still further example, UI elements (e.g., UI elements 122) for receiving a request to schedule such a service at block 802 is depicted in FIG. 9C. Again, in another example, UI elements (e.g., UI elements 122) for receiving a request to schedule such a service at block 802 is depicted in FIG. 9G.

Continuing to block 804 and block 806. At block 804 and block 806 device 100 sends a request for veterinary service availability to server 302 and server 302 receives the request. Likewise, technique 800 can include block 808 and block 810 where server 302 sends an information element including indications of appointment availability to device 100 and device 100 receives the information element. Furthermore, at block 810 processor 102 can execute instructions 120 to generate UI elements 122 (e.g., including text input boxes, check boxes, selection bubbles, drop down boxes, or the like) for display on display 106 to provide an indication of the availability (e.g., as received at block 810, or the like). Responsive to displaying UI elements 122, processor 102 can execute instructions 120 to receive a request for the veterinary service, such as, a virtual veterinary service (e.g., video consultation, or the like) or an in-home veterinary service at an available time via UI elements 122. Example UI elements, which can be UI elements 122, for receiving a request to schedule such a service at block 802 is depicted in FIG. 9D. As another example UI elements, which can be UI elements 122, for receiving a request to schedule such a service at block 802 is depicted in FIG. 9H.

Continuing to block 812 device 100 can create the appointment. For example, processor 102 can execute instructions 120 to receive a request to complete scheduling of the appointment. As a specific example, processor 102 can execute instructions 120 to generate UI elements 122 (e.g., including text input boxes, check boxes, selection bubbles, drop down boxes, or the like) for display on display 106. Responsive to displaying UI elements 122, processor 102 can execute instructions 120 to receive a request to complete the veterinary service, such as, a virtual veterinary service (e.g., video consultation, or the like) or an in-home veterinary service input via UI elements 122. Example UI elements, which can be UI elements 122, for receiving a request to complete scheduling the service at block 802 is depicted in FIG. 9E. As another example UI elements, which can be UI elements 122, for receiving a request to complete scheduling the service at block 802 is depicted in FIG. 9I.

Continuing to block 814 and block 816. At block 814 and block 816 device 100 and server 302 confirm the appointment. For example, processor 102 can execute instructions 120 to send an information element to server 302 including an indication of the confirmed appointment at block 814 and server 302 can receive the information element at block 816. Furthermore, at block 814 processor 102 can execute instructions 120 to generate UI elements 122 (e.g., including text input boxes, check boxes, selection bubbles, drop down boxes, or the like) for display on display 106 to provide an indication of the confirmed appointment. Example UI elements, which can be UI elements 122, for providing an appointment confirmation at block 814 is depicted in FIG. 9E. As another example UI elements, which can be UI elements 122, for confirming an appointment at block 814 is depicted in FIG. 9J.

Technique 800 can also include block 818 and block 820. At blocks block 818 and block 820, device 200 sends a request for calendar 228 to server 302 and server 302 receives the request. Likewise, technique 800 can include block 822 and block 824 where server 302 sends an information element including indications of calendar 228 to device 200 and device 200 receives the information element.

FIG. 9A depicts UI elements 900a which can be generated by a computing device to facilitate scheduling remote, virtual, in-home, or hybrid virtual-in-home veterinary services. For example, UI elements 900a includes a number of items 902, such as, item 902a and item 902b. As depicted, items 902 can be various display or export items (e.g., text display elements, image display elements, buttons, etc.) for receiving a request to schedule such an appointment. Processor 102 of device 100, in executing instructions 120, can generate UI elements 122 to include UI elements 900a and cause UI elements 900a to be displayed on display 106.

FIG. 9B depicts UI elements 900b which can be generated by a computing device to facilitate scheduling remote, virtual, in-home, or hybrid virtual-in-home veterinary services. For example, UI elements 900b includes a number of items 904, such as, item 904a, item 904b, and item 904c. As depicted, items 904 can be various display or export items (e.g., text display elements, image display elements, buttons, etc.) for receiving a request to schedule such an appointment for a particular pet. Processor 102 of device 100, in executing instructions 120, can generate UI elements 122 to include UI elements 900b and cause UI elements 900b to be displayed on display 106.

FIG. 9C depicts UI elements 900c which can be generated by a computing device to facilitate scheduling remote, virtual, in-home, or hybrid virtual-in-home veterinary services. For example, UI elements 900c includes a number of items 906, such as, item 906a and item 906b. As depicted, items 906 can be various display or export items (e.g., text display elements, image display elements, buttons, etc.) for receiving a request to schedule such an appointment on a particular day. Processor 102 of device 100, in executing instructions 120, can generate UI elements 122 to include UI elements 900c and cause UI elements 900c to be displayed on display 106.

FIG. 9D depicts UI elements 900d which can be generated by a computing device to facilitate scheduling remote, virtual, in-home, or hybrid virtual-in-home veterinary services. For example, UI elements 900d includes a number of items 908, such as, item 908a, item 908b, item 908c, and item 908d. As depicted, items 908 can be various display or export items (e.g., text display elements, image display elements, buttons, etc.) for receiving a request to schedule such an appointment in an available appointment window. Processor 102 of device 100, in executing instructions 120, can generate UI elements 122 to include UI elements 900d and cause UI elements 900d to be displayed on display 106.

FIG. 9E depicts UI elements 900e which can be generated by a computing device to facilitate scheduling remote, virtual, in-home, or hybrid virtual-in-home veterinary services. For example, UI elements 900e includes a number of items 910, such as, item 910a, item 910b, item 910c, item 910d, item 910e, and item 910e. As depicted, items 910 can be various display or export items (e.g., text display elements, image display elements, buttons, etc.) for confirming a request to schedule such an appointment. Processor 102 of device 100, in executing instructions 120, can generate UI elements 122 to include UI elements 900e and cause UI elements 900e to be displayed on display 106.

FIG. 9F depicts UI elements 900f which can be generated by a computing device to facilitate scheduling remote, virtual, in-home, or hybrid virtual-in-home veterinary services. For example, UI elements 900f includes a number of items 912, such as, item 912a, item 912b, item 912c, item 912d, and item 912e. As depicted, items 912 can be various display or export items (e.g., text display elements, image display elements, buttons, etc.) for confirming a request to schedule such an appointment. Processor 102 of device 100, in executing instructions 120, can generate UI elements 122 to include UI elements 900f and cause UI elements 900f to be displayed on display 106.

FIG. 9G depicts UI elements 900g which can be generated by a computing device to facilitate scheduling remote, virtual, in-home, or hybrid virtual-in-home veterinary services. For example, UI elements 900g includes a number of items 914, such as, item 914a, item 914b, item 914c, item 914d, item 914e, item 914f, and item 914g. As depicted, items 914 can be various display or export items (e.g., text display elements, image display elements, buttons, etc.) for receiving a request to schedule such an appointment for a particular pet. Processor 102 of device 100, in executing instructions 120, can generate UI elements 122 to include UI elements 900g and cause UI elements 900g to be displayed on display 106.

FIG. 9H depicts UI elements 900h which can be generated by a computing device to facilitate scheduling remote, virtual, in-home, or hybrid virtual-in-home veterinary services. For example, UI elements 900h includes a number of items 916, such as, item 916a and item 916b. As depicted, items 916 can be various display or export items (e.g., text display elements, image display elements, buttons, etc.) for receiving a request to schedule such an appointment in an available appointment window. Processor 102 of device 100, in executing instructions 120, can generate UI elements 122 to include UI elements 900h and cause UI elements 900h to be displayed on display 106.

FIG. 9I depicts UI elements 900i which can be generated by a computing device to facilitate scheduling remote, virtual, in-home, or hybrid virtual-in-home veterinary services. For example, UI elements 900i includes a number of items 918, such as, item 918aa, item 918b, item 918c, item 918d, item 918e, item 918f, and item 918g. As depicted, items 918 can be various display or export items (e.g., text display elements, image display elements, buttons, etc.) for confirming a request to schedule such an appointment. Processor 102 of device 100, in executing instructions 120, can generate UI elements 122 to include UI elements 900i and cause UI elements 900i to be displayed on display 106.

FIG. 9J depicts UI elements 900j which can be generated by a computing device to facilitate scheduling remote, virtual, in-home, or hybrid virtual-in-home veterinary services. For example, UI elements 900f includes a number of items 920, such as, item 920a, item 920b, item 920c, item 920d, and item 920e. As depicted, items 920 can be various display or export items (e.g., text display elements, image display elements, buttons, etc.) for confirming a request to schedule such an appointment. Processor 102 of device 100, in executing instructions 120, can generate UI elements 122 to include UI elements 900j and cause UI elements 900j to be displayed on display 106.

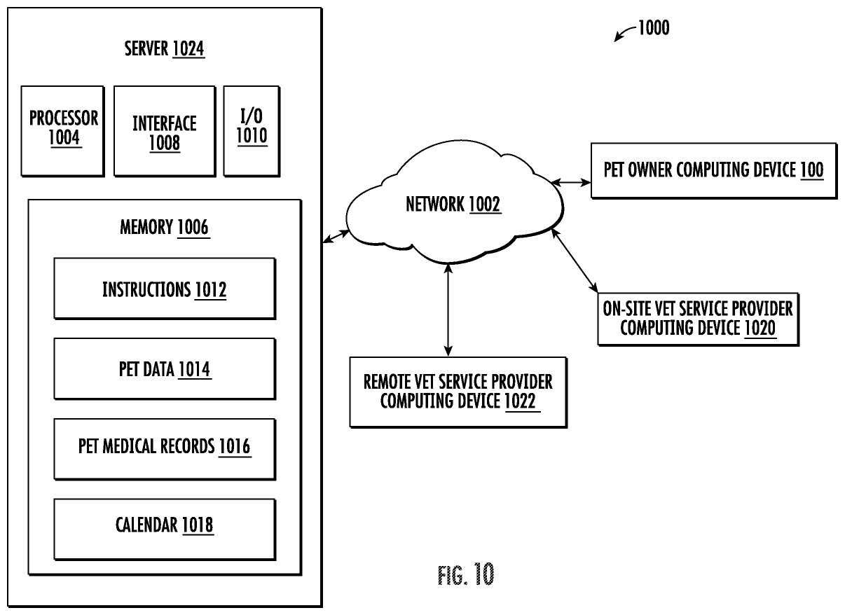

FIG. 10 illustrates an example system 1000 including device 100 and server 1024. Additionally, system 1000 includes multiple vet service provider computing devices. For example, system 1000 includes on-site vet service provider computing device 1020 and remote vet service provider computing device 1022. In general, device 1020 and device 1022 can be like device 200 of FIG. 2. In system 1000, device 100, device 1020, device 1022, and server 1024 are communicatively coupled via network 1002. Network 1002 could be, for example, a local area network (LAN), a wide area network (WAN), or a cellular network (e.g., LTE, 3GPP, or the like). In some embodiments, network 1002 could include the Internet.

The server 1024 can include a processor 1004, memory 1006, interface 1008, and I/O components 1010. The memory 1006 may store instructions 1012, pet data 1014, and calendar 1018. With some examples, processor 1004 can execute instructions 1012 to generate receive and/or provide pet data 1014 and pet medical records 1016. Additionally, processor 1004 can execute instructions 1012 to coordinate scheduling of a remote, virtual, in-home, and/or hybrid virtual-in-home veterinary service via interaction with device 100. Likewise, processor 1004 can execute instructions 1012 to provide details (e.g., via calendar 1018) of a scheduled remote, virtual, in-home, and/or hybrid virtual-in-home veterinary service to device 200. Server 302 within system 1000 can be used to facilitate other embodiments of the present disclosure, such as, for example, a remote, virtual in-home, and/or hybrid virtual-in-home veterinary appointments. These, and other embodiments, are described in greater detail below.

In particular, processor 1004 can execute instructions 1012 to facilitate a hybrid virtual-in-home veterinarian visit. For example, processor 1004 can execute instructions 1012 to provide pet data 1014 (e.g., comprising pet data 124 and pet data 224) to device 1020 and/or device 1022. Additionally, processor 1004 can execute instructions 1012 to provide pet medical records 1016 (e.g., comprising pet medical records 126 and pet medical records 226) to device 1020 and/or device 1022. Likewise, processor 1004 can execute instructions 1012 to facilitate video conferencing or remote attendance by a veterinarian via device 1022 to an in-home veterinarian visit conducted by a nurse via device 1020.

For example, during operation, a nurse can use on-site vet service provider computing device 1020 to receive pet data 224, update pet data 224, receive pet medical records 226, and/or update pet medical records 226 as part of conducting a hybrid virtual-in-home veterinary visit. In particular processor 202 can execute instructions 220 to receive pet data 224, update pet data 224, receive pet medical records 226, and/or update pet medical records 226 as part of conducting a hybrid virtual-in-home veterinary visit as described herein. Likewise, processor 202 can execute instructions 220 to conduct a virtual meeting with a veterinarian (e.g., via remote vet service provider computing device 1022) during a hybrid virtual-in-home visit.

Additionally, during operation, a veterinarian can use remote vet service provider computing device 1022 to receive pet data 224, update pet data 224, receive pet medical records 226, and/or update pet medical records 226 as part of conducting a hybrid virtual-in-home veterinary visit. In particular processor 202 can execute instructions 220 to receive pet data 224, update pet data 224, receive pet medical records 226, and/or update pet medical records 226 as part of conducting a hybrid virtual-in-home veterinary visit as described herein. Likewise, processor 202 can execute instructions 220 to attend a virtual meeting with a nurse (e.g., via on-site vet service provider computing device 1020) during a hybrid virtual-in-home visit.

Furthermore, processor 1004 can execute instructions 1012 to facilitate a hybrid virtual-in-home veterinarian visit. For example, during operation, a nurse can use on-site vet service provider computing device 1020 to receive pet data 224, update pet data 224, receive pet medical records 226, and/or update pet medical records 226 as part of conducting a hybrid virtual-in-home veterinary visit. In particular processor 202 can execute instructions 220 to receive pet data 224, update pet data 224, receive pet medical records 226, and/or update pet medical records 226 as part of conducting a hybrid virtual-in-home veterinary visit as described herein. Likewise, processor 202 can execute instructions 220 to conduct a virtual meeting with a veterinarian (e.g., via remote vet service provider computing device 1022) during a hybrid virtual-in-home visit.

In some examples, processor 1004 can execute instructions 1012 to record a remote, virtual, or hybrid virtual-in-home veterinary exam and save the exam to memory 1006. In a further example, processor 1004 can execute instructions 1012 to record and save indications of sensor data 128 and/or sensor data 230 and save such sensor data to memory 1006. For example, processor 1004 can execute instructions 1012 to add such recordings or sensor data to pet medical records 1016.

Processor 1004 can execute instructions 1012 to further identify audio and/or speech from recording or live veterinary exams (e.g., remote, virtual, hybrid virtual-in-home, etc.), transcribe the speed, and add save the transcript to memory 1006. For example, processor 1004 can execute instructions 1012 to add such transcriptions to pet medical records 1016.

With some examples, the processor 1004 may include circuity or processor logic, such as, for example, any of a variety of commercial processors. In some examples, the processor 1004 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 1004 may include graphics processing portions and may include dedicated memory, multiple-threaded processing and/or some other parallel processing capability.

Memory 1006 may include logic, a portion of which includes arrays of integrated circuits, forming non-volatile memory to persistently store data or a combination of non-volatile memory and volatile memory. It is to be appreciated, that the memory 1006 may be based on any of a variety of technologies. In particular, the arrays of integrated circuits included in memory 1006 may be arranged to form one or more types of memory, such as, for example, dynamic random access memory (DRAM), NAND memory, NOR memory, or the like.

Interface 1008 may include logic and/or features to support a communication interface. For example, the interface 1008 may include one or more interfaces that operate according to various communication protocols or standards to communicate over direct or network communication links. Direct communications may occur via use of communication protocols or standards described in one or more industry standards (including progenies and variants). For example, the interface 1008 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, interface 1008 can be a network interface arranged to couple server 302 to a network (e.g., network 1002).

As noted, system 1000 can facilitate numerous embodiments to provide remote, virtual, in-home, and/or hybrid virtual-in-home veterinary medicine. Operation and features of system 1000 are described with reference other figures depicted and described herein, such as, for example, FIG. 4.

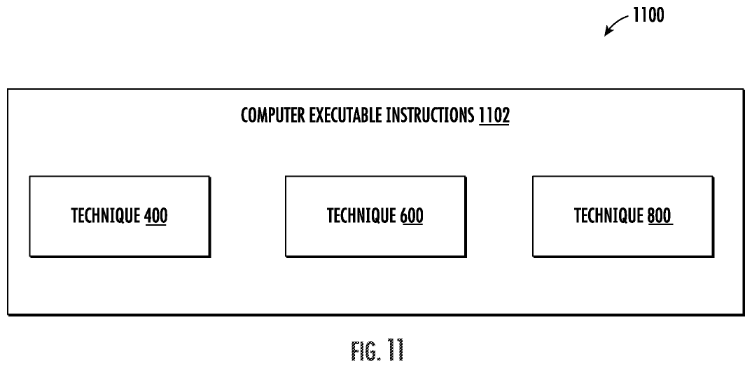

FIG. 11 illustrates computer-readable storage medium 1100. Computer-readable storage medium 1100 may comprise any non-transitory computer-readable storage medium or machine-readable storage medium, such as an optical, magnetic or semiconductor storage medium. In various embodiments, computer-readable storage medium 1100 may comprise an article of manufacture. In some embodiments, computer-readable storage medium 1100 may store computer executable instructions 1102 with which circuitry (e.g., processor 102, processor 202, processor 306, processor 1004, etc.) can execute. For example, computer executable instructions 1102 can include instructions to implement operations described with respect to technique 400, technique 600, and/or technique 800. Examples of computer-readable storage medium 1100 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1102 may include any suitable type of code, such as source code, compiled code, interpreted code, executable code, static code, dynamic code, object-oriented code, visual code, and the like.

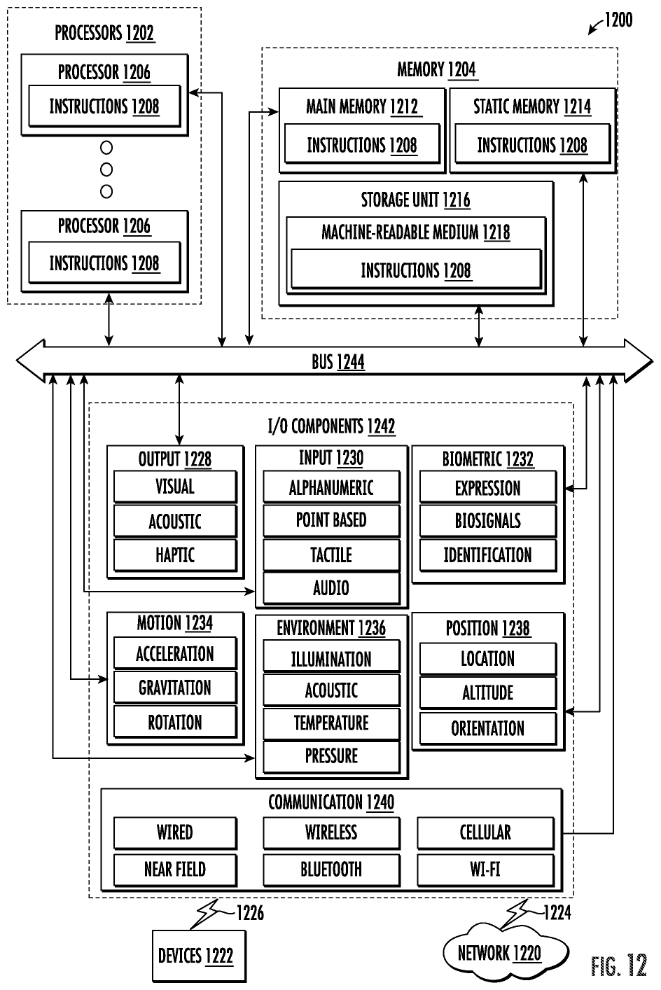

FIG. 12 illustrates a diagrammatic representation of a machine 1200 in the form of a computer system within which a set of instructions may be executed for causing the machine to perform any one or more of the methodologies discussed herein. More specifically, FIG. 12 shows a diagrammatic representation of the machine 1200 in the example form of a computer system, within which instructions 1208 (e.g., software, a program, an application, an applet, an app, or other executable code) for causing the machine 1200 to perform any one or more of the methodologies discussed herein may be executed. For example the instructions 1208 may cause the machine 1200 to execute technique 400 of FIG. 4, technique 600 of FIG. 6, and/or technique 800 of FIG. 8, or the like. More generally, the instructions 1208 may cause the machine 1200 to facilitate remote, virtual, in-home, or hybrid virtual-in-home veterinary visits as described herein.

The instructions 1208 transform the general, non-programmed machine 1200 into a particular machine 1200 programmed to carry out the described and illustrated functions in a specific manner. In alternative embodiments, the machine 1200 operates as a standalone device or may be coupled (e.g., networked) to other machines. In a networked deployment, the machine 1200 may operate in the capacity of a server machine or a client machine in a server-client network environment, or as a peer machine in a peer-to-peer (or distributed) network environment. The machine 1200 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1208, sequentially or otherwise, that specify actions to be taken by the machine 1200. Further, while only a single machine 1200 is illustrated, the term “machine” shall also be taken to include a collection of machines 200 that individually or jointly execute the instructions 1208 to perform any one or more of the methodologies discussed herein.

The machine 1200 may include processors 1202, memory 1204, and I/O components 1242, which may be configured to communicate with each other such as via a bus 1244. In an example embodiment, the processors 1202 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1206 and a processor 1210 that may execute the instructions 1208. The term “processor” is intended to include multi-core processors that may comprise two or more independent processors (sometimes referred to as “cores”) that may execute instructions contemporaneously. Although FIG. 12 shows multiple processors 1202, the machine 1200 may include a single processor with a single core, a single processor with multiple cores (e.g., a multi-core processor), multiple processors with a single core, multiple processors with multiples cores, or any combination thereof.

The memory 1204 may include a main memory 1212, a static memory 1214, and a storage unit 1216, both accessible to the processors 1202 such as via the bus 1244. The main memory 1204, the static memory 1214, and storage unit 1216 store the instructions 1208 embodying any one or more of the methodologies or functions described herein. The instructions 1208 may also reside, completely or partially, within the main memory 1212, within the static memory 1214, within machine-readable medium 1218 within the storage unit 1216, within at least one of the processors 1202 (e.g., within the processor's cache memory), or any suitable combination thereof, during execution thereof by the machine 1200.

The I/O components 1242 may include a wide variety of components to receive input, provide output, produce output, transmit information, exchange information, capture measurements, and so on. The specific I/O components 1242 that are included in a particular machine will depend on the type of machine. For example, portable machines such as mobile phones will likely include a touch input device or other such input mechanisms, while a headless server machine will likely not include such a touch input device. It will be appreciated that the I/O components 1242 may include many other components that are not shown in FIG. 12. The I/O components 1242 are grouped according to functionality merely for simplifying the following discussion and the grouping is in no way limiting. In various example embodiments, the I/O components 1242 may include output components 1228 and input components 1230. The output components 1228 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1230 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1242 may include biometric components 1232, motion components 1234, environmental components 1236, or position components 1238, among a wide array of other components. For example, the biometric components 1232 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1234 may include acceleration sensor components (e.g., accelerometer), gravitation sensor components, rotation sensor components (e.g., gyroscope), and so forth. The environmental components 1236 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1238 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1242 may include communication components 1240 operable to couple the machine 1200 to a network 1220 or devices 1222 via a coupling 1224 and a coupling 1226, respectively. For example, the communication components 1240 may include a network interface component or another suitable device to interface with the network 1220. In further examples, the communication components 1240 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1222 may be another machine or any of a wide variety of peripheral devices (e.g., a peripheral device coupled via a USB).

Moreover, the communication components 1240 may detect identifiers or include components operable to detect identifiers. For example, the communication components 1240 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1240, such as location via Internet Protocol (IP) geolocation, location via Wi-Fi® signal triangulation, location via detecting an NFC beacon signal that may indicate a particular location, and so forth.

The various memories (i.e., memory 1204, main memory 1212, static memory 1214, and/or memory of the processors 1202) and/or storage unit 1216 may store one or more sets of instructions and data structures (e.g., software) embodying or utilized by any one or more of the methodologies or functions described herein. These instructions (e.g., the instructions 1208), when executed by processors 1202, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1220 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1220 or a portion of the network 1220 may include a wireless or cellular network, and the coupling 1224 may be a Code Division Multiple Access (CDMA) connection, a Global System for Mobile communications (GSM) connection, or another type of cellular or wireless coupling. In this example, the coupling 1224 may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1208 may be transmitted or received over the network 1220 using a transmission medium via a network interface device (e.g., a network interface component included in the communication components 1240) and utilizing any one of a number of well-known transfer protocols (e.g., hypertext transfer protocol (HTTP)). Similarly, the instructions 1208 may be transmitted or received using a transmission medium via the coupling 1226 (e.g., a peer-to-peer coupling) to the devices 1222. The terms “transmission medium” and “signal medium” mean the same thing and may be used interchangeably in this disclosure. The terms “transmission medium” and “signal medium” shall be taken to include any intangible medium that is capable of storing, encoding, or carrying the instructions 1208 for execution by the machine 1200, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

What is claimed is:

1. A computer-implemented method, comprising:
determining an appointment type for a veterinary service from a plurality of appointment types, the plurality of appointment types comprising a virtual appointment and an in-home appointment;
identifying an animal to associate with the veterinary service;
identifying a date for the veterinary service;
determining a set of providers for the veterinary service based on the appointment type and the animal associated with the veterinary service;
determining, for each provider in the set of providers, a set of available times on the date for the veterinary service;
generating a first information element with indications of availability for the veterinary service based on the set of available times for each provider in the set of providers;
transmitting the first information element to a client computing device; and
scheduling the veterinary service on the date with a first provider in the set of providers and for a first time in the set of available times based on a second information element received from the client computing device in response to the first information element.

2. The computer-implemented method of claim 1, comprising:
receiving a first calendar from a first provider computing device associated with the first provider in the set of providers; and
determining the set of available times for the first provider on the date for the veterinary service based on the first calendar.

3. The computer-implemented method of claim 2, wherein scheduling the veterinary service on the date with the first provider for the first time includes transmitting an updated calendar to the first provider computing device.

4. The computer-implemented method of claim 1, wherein the plurality of appointment types include a hybrid virtual-in-home veterinary service comprising a virtual component with a virtual provider and an in-home component with an in-person provider.

5. The computer-implemented method of claim 4, comprising:
scheduling the veterinary service as an in-home appointment with the first provider; and
modifying the in-home appointment to a hybrid virtual-in-home veterinary service in response to receiving an information element from a first provider computing device associated with the first provider,
wherein the first provider comprises the in-person provider.

6. The computer-implemented method of claim 5, comprising:
receiving a second calendar from a second provider computing device associated with a second provider; and
identifying the second provider as the virtual provider based on the second calendar.

7. The computer-implemented method of claim 5, comprising:
identifying a second provider as the virtual provider, the second provider associated with a second provider computing device; and
establishing a virtual meeting between the first and second provider computing devices.

8. The computer-implemented method of claim 1, comprising:
receiving a location for the veterinary service; and
determining the set of providers for the veterinary service based on the appointment type, the animal associated with the veterinary service, and the location received for the veterinary service.

9. An apparatus, the apparatus comprising:
a processor; and
memory comprising instructions that when executed by the processor cause the processor to:
determine an appointment type for a veterinary service from a plurality of appointment types, the plurality of appointment types comprising a virtual appointment and an in-home appointment;
identify an animal to associate with the veterinary service;
identify a date for the veterinary service;
determine a set of providers for the veterinary service based on the appointment type and the animal associated with the veterinary service;
determine, for each provider in the set of providers, a set of available times on the date for the veterinary service;
generate a first information element with indications of availability for the veterinary service based on the set of available times for each provider in the set of providers;
transmit the first information element to a client computing device; and
schedule the veterinary service on the date with a first provider in the set of providers and for a first time in the set of available times based on a second information element received from the client computing device in response to the first information element.

10. The apparatus of claim 8, wherein the instructions, when executed by the processor, further cause the processor to:
receive a first calendar from a first provider computing device associated with the first provider in the set of providers; and
determine the set of available times for the first provider on the date for the veterinary service based on the first calendar.

11. The apparatus of claim 9, wherein the instructions, when executed by the processor, further cause the processor to transmit an updated calendar to the first provider computing device to schedule the veterinary service on the date with the first provider for the first time.

12. The apparatus of claim 8, wherein the plurality of appointment types include a hybrid virtual-in-home veterinary service comprising a virtual component with a virtual provider and an in-home component with an in-person provider.

13. The apparatus of claim 11, wherein the instructions, when executed by the processor, further cause the processor to:
schedule the veterinary service as an in-home appointment with the first provider; and
modify the in-home appointment to a hybrid virtual-in-home veterinary service in response to receive an information element from a first provider computing device associated with the first provider,
wherein the first provider comprises the in-person provider.

14. The apparatus of claim 12, wherein the instructions, when executed by the processor, further cause the processor to:
receive a second calendar from a second provider computing device associated with a second provider; and
identify the second provider as the virtual provider based on the second calendar.

15. The apparatus of claim 12, wherein the instructions, when executed by the processor, further cause the processor to:
identify a second provider as the virtual provider, the second provider associated with a second provider computing device; and
establish a virtual meeting between the first and second provider computing devices.

16. At least one non-transitory computer-readable medium comprising a set of instructions that, in response to being executed by a processor circuit, cause the processor circuit to:
determine an appointment type for a veterinary service from a plurality of appointment types, the plurality of appointment types comprising a virtual appointment and an in-home appointment;
identify an animal to associate with the veterinary service;
identify a date for the veterinary service;
determine a set of providers for the veterinary service based on the appointment type and the animal associated with the veterinary service;
determine, for each provider in the set of providers, a set of available times on the date for the veterinary service;
generate a first information element with indications of availability for the veterinary service based on the set of available times for each provider in the set of providers;
transmit the first information element to a client computing device; and
schedule the veterinary service on the date with a first provider in the set of providers and for a first time in the set of available times based on a second information element received from the client computing device in response to the first information element.

17. The at least one non-transitory computer-readable medium of claim 15, wherein the set of instructions, in response to execution by the processor circuit, further cause the processor circuit to:
receive a first calendar from a first provider computing device associated with the first provider in the set of providers; and
determine the set of available times for the first provider on the date for the veterinary service based on the first calendar.

18. The at least one non-transitory computer-readable medium of claim 16, wherein the set of instructions, in response to execution by the processor circuit, further cause the processor circuit to transmit an updated calendar to the first provider computing device to schedule the veterinary service on the date with the first provider for the first time.

19. The at least one non-transitory computer-readable medium of claim 15, wherein the plurality of appointment types include a hybrid virtual-in-home veterinary service comprising a virtual component with a virtual provider and an in-home component with an in-person provider.

20. The at least one non-transitory computer-readable medium of claim 18, wherein the set of instructions, in response to execution by the processor circuit, further cause the processor circuit to:
schedule the veterinary service as an in-home appointment with the first provider; and
modify the in-home appointment to a hybrid virtual-in-home veterinary service in response to receive an information element from a first provider computing device associated with the first provider,
wherein the first provider comprises the in-person provider.

21. The at least one non-transitory computer-readable medium of claim 19, wherein the set of instructions, in response to execution by the processor circuit, further cause the processor circuit to:
identify a second provider as the virtual provider, the second provider associated with a second provider computing device; and
establish a virtual meeting between the first and second provider computing devices.
